(19) United States
(12) Patent Application Publication
(10) Pub. No.: US 2026/0249064 A1
(43) Pub. Date: Aug. 27, 2026

(54) CIRCULATION SUPPORT DEVICES, SYSTEMS, AND METHODS

(71) Applicant: BOSTON SCIENTIFIC SCIMED, INC., MAPLE GROVE, MN (US)

(72) Inventors: Chris Cullen, Galway (IE); Tim O'Connor, Claregalway (IE); Neil O'Connor, Oranmore (IE); Gavin O'Brien, Sutton (IE); Benjamin Carl Breidall, Eden Prairie, MN (US)

(73) Assignee: BOSTON SCIENTIFIC SCIMED, INC., MAPLE GROVE, MN (US)

(21) Appl. No.: 19/546,681

(22) Filed: Feb. 23, 2026

Related U.S. Application Data
(60) Provisional application No. 63/762,374, filed on Feb. 24, 2025.

(51) Int. Cl. A61M 39/02; A61F 2/856; A61M 60/165; A61M 60/865

(52) U.S. Cl. CPC A61M 39/0247; A61F 2/856; A61M 60/165; A61M 60/865; A61F 2220/0008; A61F 2250/0098; A61M 2205/0238; A61M 2205/32; A61M 2210/04; A61M 2210/12

(57) ABSTRACT

A vascular access device may include an elongate tubular frame and a tubular extension extending radially outward from an outer surface of the elongate tubular frame. The elongate tubular frame may define a first opening at a first end of the elongate tubular frame, a second opening at a second end of the elongate tubular frame, and a third opening at a location between the first end and the second end. A first lumen extending through the elongate tubular frame may be in communication with the first opening, the second opening, and the third opening. The tubular extension may be formed from a flexible polymer and define a second lumen in fluid communication with the first lumen at the third opening. A coating may extend along an inner surface of the elongate tubular frame and define the first lumen.

### CROSS-REFERENCE TO RELATED APPLICATION

This application claims the benefit of U.S. Patent Application Serial No. 63/762,374, filed February 24, 2025, entitled " CIRCULATION SUPPORT DEVICES, SYSTEMS, AND METHODS”, which is incorporated by reference herein in its entirety.

### TECHNICAL FIELD

The present disclosure pertains to cardiac devices and systems. More specifically, the present disclosure relates to devices, systems, and methods of and/or for delivering cardiac devices and systems.

### BACKGROUND

A wide variety of medical devices have been developed for medical use including, for example, medical devices utilized to provide transient support in patients with compromised heart function or cardiac output. Generally, such devices, which may be referred to as percutaneous circulatory support devices or percutaneous blood pumps, are delivered to a patient's heart through the vasculature of the patient via a femoral access site. However, there is an ongoing need to provide alternative configurations of vascular access devices for use with percutaneous blood pumps, and methods of gaining vascular access and advancement of the percutaneous blood pump into a patient’s vasculature.

### BRIEF SUMMARY

This disclosure provides design, material, manufacturing method, and use alternatives for medical devices, including cardiac devices and devices used therewith.

A first example may include a vascular access device comprising an elongate tubular frame defining a first opening at a first end of the elongate tubular frame, a second opening at a second end of the elongate tubular frame, and a third opening at a location between the first end and the second end, wherein a first lumen extending through the elongate tubular frame is in communication with the first opening, the second opening, and the third opening and a tubular extension formed from a flexible polymer and defining a second lumen in fluid communication with the first lumen at the third opening, the tubular extension extends radially outward from an outer surface of the elongate tubular frame.

Alternatively or additionally to any of the examples above, the elongate tubular frame may be a stent.

Alternatively or additionally to any of the examples above, the elongate tubular frame may be a self-expanding elongate tubular frame.

Alternatively or additionally to any of the examples above, the vascular access device may include a coating extending along an inner surface of the elongate tubular frame and defining the first lumen extending through the elongate tubular frame.

Alternatively or additionally to any of the examples above, the coating and the tubular extension may be monolithically formed from a single material.

Alternatively or additionally to any of the examples above, the coating and the tubular extension may be formed from silicone.

Alternatively or additionally to any of the examples above, the vascular access device may include a radiopaque marker fixed relative to the third opening.

Alternatively or additionally to any of the examples above, the elongate tubular frame may be formed from a radiopaque material.

Alternatively or additionally to any of the examples above, the tubular extension may have a length in a range of 25.0 millimeters (mm) to 125.0 mm.

Alternatively or additionally to any of the examples above, one or both of the first lumen and the second lumen may have a maximum diameter in a range 5.0 mm to 8 mm.

Alternatively or additionally to any of the examples above, a center of the third opening may be equidistance from the first end of the elongate tubular frame and the second end of the elongate tubular frame.

In a further example, a method of using a vascular access device may include advancing the vascular access device through a vessel in fluid communication with and proximal of an axillary artery, the vascular access device having an elongate tubular frame and a tubular extension extending radially outward from an outer surface of the elongate tubular frame, deploying the elongate tubular frame within the axillary artery with the tubular extension extending along the elongate tubular frame, creating an opening through skin and at a location proximate the tubular extension, and extending the tubular extension through the opening.

Alternatively or additionally to any of the examples above, the method may further include orienting the tubular extension at a desired location relative to an intended location of the opening through the skin and the axillary artery.

Alternatively or additionally to any of the examples above, the method may further include delivering a blood pump to a heart through the tubular extension and the elongate tubular frame.

Alternatively or additionally to any of the examples above, the method may further include percutaneously accessing the vessel in fluid communication with and proximal of the axillary artery.

Alternatively or additionally to any of the examples above, the vessel may be a radial artery.

Alternatively or additionally to any of the examples above, the tubular extension may be formed from a flexible polymer material.

In a further example, a stent assembly may include an elongate tubular frame having an inner surface and an outer surface, the elongate tubular frame defining a first opening at a first end of the elongate tubular frame, a second opening at a second end of the elongate tubular frame, and a third opening extending through the elongate tubular frame at a location between the first end and the second end, a coating extending along the inner surface of the elongate tubular frame and defining a first lumen extending through the elongate tubular frame, and a tubular extension defining a second lumen in fluid communication with the first lumen at the third opening.

Alternatively or additionally to any of the examples above, the coating extending along the inner surface of the elongate tubular frame and the tubular extension are continuously formed from a flexible polymer material.

Alternatively or additionally to any of the examples above, the elongate tubular frame may have a minimum outer diameter that is less than 5.0 mm and a maximum outer diameter that is less than 8.0 mm.

The above summary of some configurations is not intended to describe each disclosed configuration or every implementation of the present disclosure. The Figures, and Detailed Description, which follow, more particularly illustrate some of these configurations.

### BRIEF DESCRIPTION OF THE DRAWINGS

The disclosure may be more completely understood in consideration of the following detailed description in connection with the accompanying drawings, in which:

FIG. 1 is a schematic perspective view of an illustrative percutaneous circulatory support system;

FIG. 2 is a schematic partial cross-sectional view of anatomy and a schematic side view of an illustrative percutaneous circulatory support system within the anatomy;

FIG. 3 is a schematic cross-sectional view of a portion of an introducer sheath within a blood vessel and a portion of an illustrative percutaneous circulatory support system in the introducer sheath and the blood vessel;

FIG. 4 is a schematic perspective view of an illustrative vascular access device;

FIG. 5 is a schematic side view of an illustrative vascular access device;

FIG. 6A is a schematic cross-sectional view of a portion of a delivery system within a blood vessel and a vascular access device along the delivery system, with the delivery system and the vascular access device in a delivery configuration;

FIG. 6B is a schematic cross-section view of the portion of a delivery system within a blood vessel and the vascular access device along the delivery system depicted in FIG. 6A, with the delivery system and the vascular access device in a deployed configuration;

FIG. 7 is a schematic view of an illustrative delivery path through patient anatomy for positioning a vascular access device at a target location;

FIG. 8 is a schematic box diagram of an illustrative method of using the vascular access device; and

FIGS. 9A-9E schematically depict views illustrating a portion of an illustrative technique for positioning the vascular access device at a target location.

While the disclosure is amenable to various modifications and alternative forms, specifics thereof have been shown by way of example in the drawings and will be described in detail. The intention is not to limit the disclosure to the particular configurations described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the term “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “a configuration”, “some configurations”, “other configurations”, etc., indicate that the configuration described may include one or more particular features, structures, and/or characteristics. However, such recitations do not necessarily mean that all configurations include the particular features, structures, and/or characteristics. Additionally, when particular features, structures, and/or characteristics are described in connection with one configuration, such features, structures, and/or characteristics may also be used in connection with other configurations whether or not explicitly described unless clearly stated to the contrary.

The following detailed description should be read with reference to the drawings in which similar structures in different drawings are numbered the same. The drawings, which are not necessarily to scale, depict illustrative configurations and are not intended to limit the scope of the disclosure. Additionally, it should be noted that in any given figure, some features may not be shown, or may be shown schematically, for clarity and/or simplicity. Additional details regarding some components and/or method steps may be illustrated in other figures in greater detail. The devices and/or methods disclosed herein may provide a number of desirable features and benefits as described in more detail below.

A variety of circulatory assist or support devices are known for assisting or replacing a pumping function of a heart in a patient with severe heart failure and/or other cardiac conditions. Circulatory support devices may be configured to treat patients with cardiogenic shock, myocardial infarction, acutely decompensated heart failure, and/or other heart related conditions. Additionally or alternatively circulatory support systems or devices may support a patient during percutaneous coronary interventions and/or other procedures.

Example cardiac circulatory support systems or devices include, but are not limited to, ventricular assist devices (VADs), total artificial hearts, intra-aortic balloon pumps (IABP), and extracorporeal membrane oxygenation (ECMO) devices. Example VADs include left ventricular assist devices (LVADs), right ventricular assist devices (RVADs), and biventricular assist devices (BiVADs). A further illustrative VAD is a percutaneous ventricular assist device (PVAD), which may be inserted into a ventricle (e.g., a left ventricle or a right ventricle) of a heart of a patient via delivery through an artery (e.g., femoral, axillary, etc.) or vein and/or other suitable vasculature to the ventricle. A PVAD may be placed at a desired location of anatomy of a patient via percutaneous access and delivery, which may enable the PVAD to be used in emergency medicine, a cath lab, and/or other surgical and/or non-surgical settings.

In some examples, percutaneous circulatory support systems may include a distal end having a flexible cannula that is configured to be inserted into a heart of a patient. A housing (e.g., a rigid housing) that may house one or more pumping components may extend in a proximal direction from a proximal end of the flexible cannula. One or more bends in the flexible cannula may direct the distal end of the flexible cannula through the aortic valve of the patient and into the left ventricle of the heart.

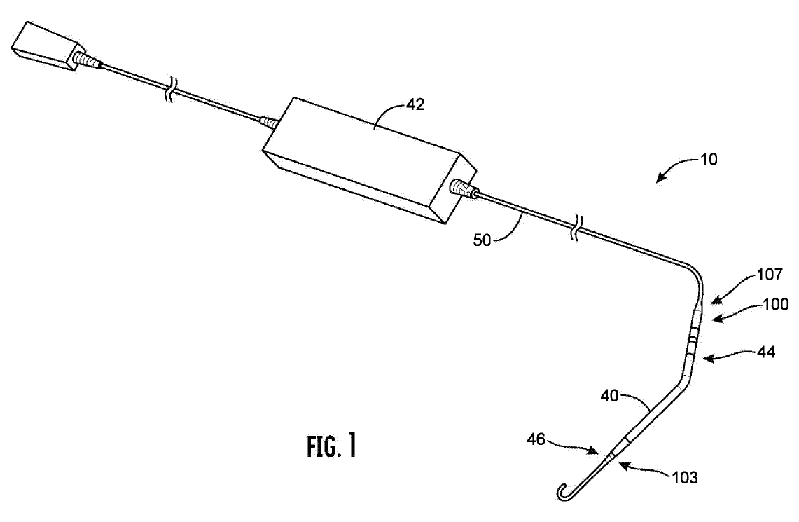

FIG. 1 depicts a schematic view of an illustrative percutaneous circulatory support system 10 (e.g., a blood pump). The system 10 may include a percutaneous support device (e.g., a PVAD, such as a blood pump 100 having a proximal end 107 and a distal end 103), a cannula 40 having a proximal end 44 and a distal end 46, an elongate shaft 50, and a delivery or guide sheath (not shown). In some examples, the system 10 may include a guidewire, but this is not required. The delivery or guide sheath, when included, may facilitate percutaneous delivery of the blood pump 100 and the cannula 40 to a target location within a patient (e.g., a target location within a heart of a patient). When positioned at a target site in a heart of a patient, the blood pump 100 may be configured to pump blood from a ventricle of the heart to vasculature of the patient.

The cannula 40 may be a component of the blood pump 100 and/or a separate component coupled with the blood pump 100. In some examples, although the cannula 40 may be discussed as extending from the blood pump 100 with the proximal end 44 of the cannula 40 at or proximate the distal end 103 of the blood pump 100, the cannula 40 may be part of the blood pump 100 (e.g., formed from a component of the blood pump 100, such as an impeller housing and/or other suitable component) with the distal end 46 of the cannula 40 at the distal end 103 of the blood pump 100, but other suitable configurations are contemplated.

The system 10 may further include a proximal housing 42 coupled with (e.g., connected to) the elongate shaft 50 (e.g., a catheter shaft and/or other suitable elongate tube), where the elongate shaft 50 may be coupled with the blood pump 100 (e.g., a distal end of the elongate shaft 50 may be coupled with the proximal end 107 of the blood pump 100) and one or more wires and/or shafts coupled with the blood pump 100 may extend proximally from the blood pump 100 through the shaft 50. In some examples, at least the proximal housing 42, the elongate shaft 50, and/or other components of the system 10 may be configured to facilitate delivering the blood pump 100 and the cannula 40 to the target location or site within the patient. The elongate shaft 50 and the proximal housing 42 may be configured to be positioned at least partially exterior of the patient when the blood pump 100 and the cannula 40 are at the target location. In some examples, the guidewire, when included, may also be used to facilitate the delivery of the blood pump 100 and/or the cannula 40.

Although not shown, the system 10 may additionally or alternatively include one or both of a starter tube and a starter tube flushing line. The starter tube, when included, may couple with a cannula delivery tool (e.g., a delivery sheath) which may be configured to receive the cannula 40 prior to and/or during delivery of the blood pump 100 and the cannula 40 to a target site.

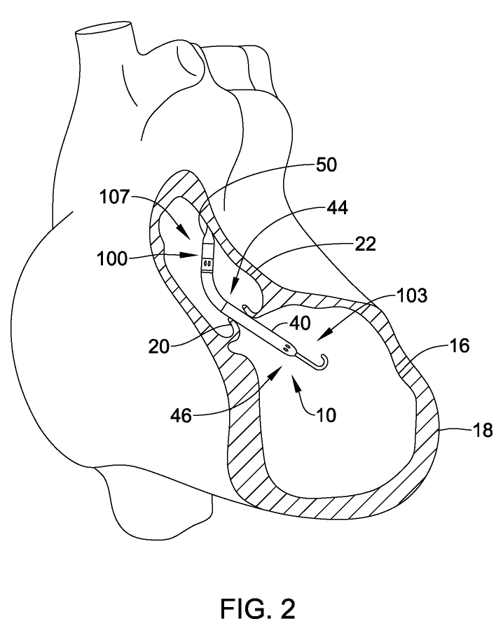

FIG. 2 depicts a schematic view of an illustrative positioning of the blood pump 100 (e.g., a percutaneous circulatory support device, such as a PVAD, etc.) in anatomy of a patient (e.g., in a left ventricle 16 in a heart 18). As depicted in FIG. 2, the blood pump 100 may be positioned with the distal end 103 thereof (e.g., when including or combined with the cannula 40) located in the left ventricle 16 of the heart 18 and the proximal end 107 of the blood pump 100 in an aorta 22, such that the blood pump 100 and the cannula 40 in combination extends across an aortic valve 20 between the left ventricle 16 and the aorta 22. With the blood pump 100 extending from the left ventricle 16 to the aorta 22 of the patient, the blood pump 100 may be configured to pump blood from the left ventricle 16 into the aorta 22 (e.g., into the ascending aorta) to assist or support blood flow circulation. Other suitable positions of the blood pump 100 relative to the anatomy of the patient are contemplated and include, but are not limited to, the distal end 103 of the blood pump 100 being positioned in or in fluid communication with a right ventricle of the heart 18 with the proximal end 107 of the blood pump 100 being positioned in a pulmonary artery.

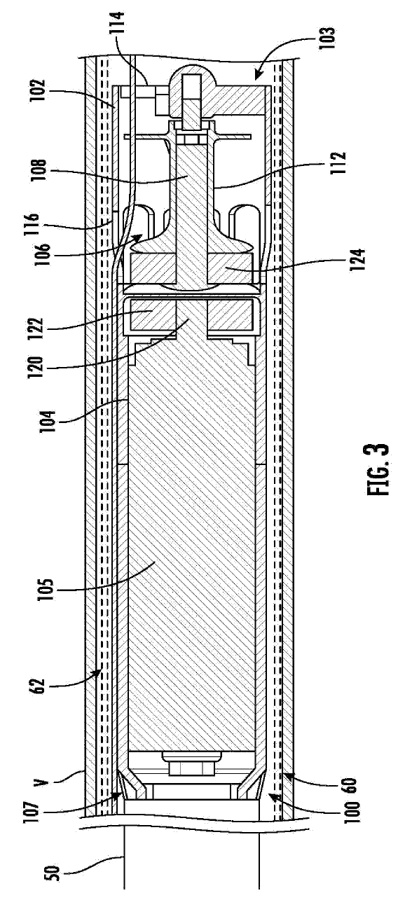

FIG. 3 depicts a schematic cross-section view of a body portion 62 of a delivery or guide sheath 60 (hereinafter “guide sheath 60”) extending into a blood vessel V with the blood pump 100 inserted into the guide sheath 60. The blood pump 100 may include and/or may be coupled with the elongate shaft 50 extending proximally from a proximal end 107 of the blood pump 100 to a location outside of the blood vessel V and the guide sheath 60. When positioning the blood pump 100 within the patient, the blood pump 100 may be advanced through the blood vessel V and positioned in or at a target location, such as a target cardiac location (e.g., in the aorta 22, across the aortic valve 20, and/or into the left ventricle 16), via the guide sheath 60. While the guide sheath 60 is illustrated in FIG. 3 with the use of the blood pump 100, in other instances the guide sheath 60 may be omitted or various other medical devices may be used in conjunction with the guide sheath 60.

The blood pump 100 may include one or more components. In some examples, the blood pump 100 may include an impeller housing 102 and a motor housing 104. The impeller housing 102 and the motor housing 104 may be integrally or monolithically constructed. Alternatively, the impeller housing 102 and the motor housing 104 may be separate components. The impeller housing 102 may carry an impeller assembly 106 therein. The impeller assembly 106 may include an impeller shaft 108 and an impeller 112 that rotates relative to the impeller housing 102 to drive or pump blood from the left ventricle 16 of the heart 18 of the patient (e.g., via the cannula 40 of or in fluid communication with the blood pump 100) to the aorta 22 or portion of the anatomy in communication with the aorta (e.g., pump blood through the blood pump 100 to an ascending aorta). In some examples, the impeller shaft 108 and the impeller 112 may be integrally formed, whereas, in other examples the impeller shaft 108 and the impeller 112 may be separate components.

Rotation of the impeller 112 may cause blood to flow from a blood inlet 114 formed on the impeller housing 102 (e.g., where the blood may enter the blood inlet 114 directly from the left ventricle and/or via the cannula 40), through the impeller housing 102, and out of a blood outlet 116 formed on the impeller housing 102. As shown in FIG. 3, the blood inlet 114 may be formed on an end portion of the impeller housing 102 and the blood outlet 116 may be formed on a side portion of the impeller housing 102. In other examples, the blood inlet 114 and/or the blood outlet 116 may be formed on or at other portions of the impeller housing 102 or other components of the system 10.

The impeller housing 102 of the impeller assembly 106 may be coupled to the cannula 40 and the cannula 40 may extend distally from the impeller housing 102 (e.g., extending distally from the distal end 103 of the blood pump 100), and the cannula 40 may receive blood from the left ventricle and deliver the blood to the blood inlet 114. Alternatively or additionally, the impeller housing 102 may be further elongated than depicted in FIG. 3 and define all of or at least part of the cannula 40 extending into the left ventricle of the patient. Other suitable configurations of the impeller housing 102 and/or the cannula 40 are contemplated.

The motor housing 104 may carry a motor 105, and the motor 105 may be configured to rotatably drive the impeller 112 relative to the impeller housing 102 when the motor 105 is actuated. In some examples, the motor 105 may rotate a drive shaft 120 coupled with a driving magnet 122. Rotation of the driving magnet 122 may cause rotation of a driven magnet 124 that may be coupled with and/or may be part of the impeller assembly 106. More specifically, in configurations incorporating the impeller shaft 108, the impeller shaft 108 and the impeller 112 may be configured to rotate with the driven magnet 124. In other configurations, the motor 105 and/or the drive shaft 120 may be coupled to the impeller assembly 106 directly and/or via other suitable components.

As discussed, the blood pump 100 may be delivered to the heart of the patient via an arterial access site. In some examples, the blood pump 100 may be delivered to the heart of the patient via a femoral access site. However, utilizing a femoral access site may limit the mobility of the patient as movement of the elongate shaft 50 (extending out of a femoral access site) as the patient moves, may result in inadvertent adjustment of a position of the blood pump 100 in the heart of the patient. As such, to allow the patient to be ambulatory with the blood pump 100 operating in the heart of the patient, it may be advantageous to utilize an access site closer to the heart of the patient and/or not at a limb of the body that moves relative to a core (e.g., relative to a chest) of the patient as the patient becomes ambulatory. In some examples, the axillary artery or subscapular artery may have a sufficient diameter to receive the blood pump and is close enough to the heart of the patient (e.g., located at the chest of the patient) to mitigate a likelihood of the elongate shaft 50 adjusting in a manner that would inadvertently adjust the position of the blood pump 100 in the heart of the patient as the patient becomes ambulatory.

The axillary artery and/or the subscapular artery may be accessed via surgical techniques. Using a surgical technique, a graft may be attached to the axillary artery or the subscapular artery and the blood pump 100 may be delivered to the heart of the patient via the axillary artery and/or the subscapular artery by inserting the blood pump 100 into the graft and through the axillary artery and/or the scapular artery. Use of the graft may facilitate providing an access site for inserting the blood pump 100 to the heart of the patient and through which the elongate shaft 50 may extend during operation of the blood pump 10, which may mitigate bleeding from the vasculature and mitigate a likelihood of infection. Further, maintaining the graft at the access site may facilitate removal of the blood pump 100 from the patient after a period of time. Delivering the blood pump 100 to the heart of the patient via the axillary artery, the subscapular artery, and/or other suitable artery proximate the heart of the patient (e.g., at an upper extremity access site) may facilitate supporting a patient utilizing the blood pump 100 in an ambulatory state for an extended period of time (e.g., up to 2 weeks and/or beyond 2 weeks).

Rather than connecting a graft to the axillary artery and/or subscapular artery via a surgical approach, a percutaneous approach may be utilized for providing a graft or access device at a desired location along the axillary artery or other suitable artery. Utilizing a percutaneous approach for placing a graft or access tube may reduce bleeding and/or mitigate infection risks at the arterial access site for inserting the blood pump 100 into the vasculature of the patient. In some examples, a vascular access device with an elongate tubular frame and a graft or tubular extension extending from the elongate tubular frame may be inserted into an arterial system of the patient (e.g., via a radial artery or other suitable arterial access site) and delivered to a target location in the axillary artery or other suitable target location. Once the elongate tubular frame and graft are delivered to the target location, a cut or incision through the skin of the patient and the wall of the axillary artery of the patient may be made and the graft or tubular extension may be extended out through the wall of the axillary artery and out of the cut or incision through the skin of the patient. The blood pump 100 may then be delivered to the heart of the patient via the graft or tubular extension accessed in a subscapular region, for example, providing access to the axillary artery of the patient.

Although the vascular access device may be discussed herein with respect to delivering the blood pump 100 to the heart of the patient, other suitable devices or systems may be delivered to the heart or other anatomy of the patient via the vascular access device. Example devices that may be delivered to the heart of the patient via the vascular access device may include, but are not limited to, replacement valves, leads for pacemakers, etc. Although the vascular access device may be discussed herein with being place at the axillary artery, the vascular access device may be placed at other suitable locations in the vasculature of the patient. Although the vascular access device may be discussed herein as being inserted into the vasculature via the radial artery, the vascular access device may be inserted into the vasculature of the patient via one or more other suitable vessels in a percutaneous manner.

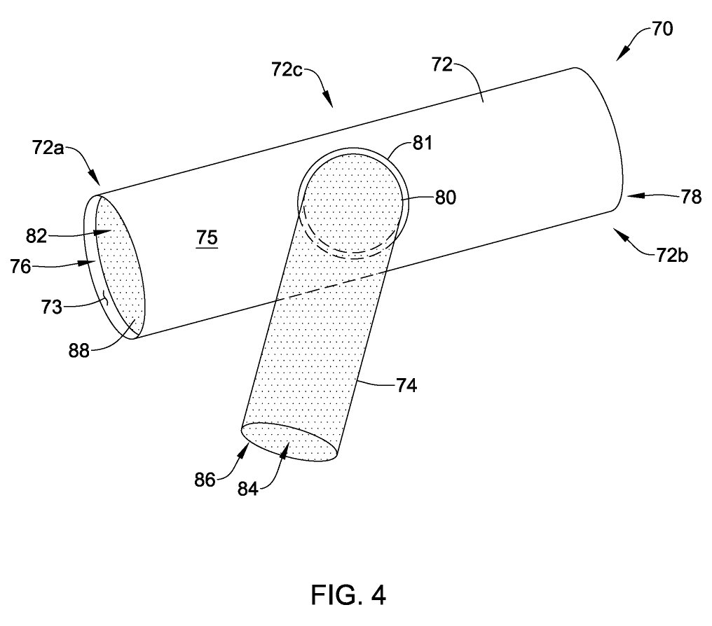

FIG. 4 depicts a schematic perspective of an illustrative vascular access device 70. In some examples, the vascular access device 70 may be configured for percutaneous placement in the vasculature of a patient. In some examples, the vascular access device 70 may be percutaneously placed at a target site or location in a vessel and accessed at the target site to receive a medical device (e.g., the blood pump 100 and/or other suitable medical device) for delivery through the vasculature to another target site or location in or in communication with the vasculature. In some example, the vascular access device 70 may be configured for placement in an axillary artery, but the vascular access device 70 may be configured for placement in additional or alternative vessels.

The vascular access device 70 may have any suitable configuration. For example, the vascular access device may include an elongate tubular frame 72 and a tubular extension 74 extending from the elongate tubular frame 72.

The elongate tubular frame 72 may include an inner surface 73, an outer surface 75, a first opening 76 at a first end 72a, a second opening 78 at a second end 72b, and an intermediate region 72c between the first end 72a and the second end 72b. A third opening 80 may be located along the intermediate region 72c of the elongate tubular frame 72 between the first end 70a and the second end 70b. Although the first opening 76, the second opening 78, and the third opening 80 are disclosed as being part of or defined by the elongate tubular frame 72, the first opening 76, the second opening 78, and the third opening 80 may be formed of or may be a part of one or more other components of the vascular access device 70.

A first lumen 82 may extend through the elongate tubular frame 72. The first lumen 82 may extend from the first end 72a to the second end 72b of the elongate tubular frame 72, such that the first lumen 82 extends from the first opening 76 to the second opening 78. In some examples, the first lumen 82 may be in fluid communication with the first opening 76, the second opening 78, and the third opening 80.

The third opening 80 may have any suitable shape. In some examples, the third opening 80 have a circular shape, as depicted for example in FIG. 4. Alternatively or additionally, the third opening 80 may have an oblong shape or other suitable shape for receiving a medical device into the first lumen 82 through the tubular extension 74.

The third opening 80 may be at any suitable location along the elongate tubular frame 72. For example, the third opening 80 may be closer to the first end 72a of the elongate tubular frame 72, closer to the second end 72b of the elongate tubular frame 72, equidistance from the first end 72a and the second end 72b of the elongate tubular frame 72, and/or the third opening 80 may be at one or more other suitable locations along the elongate tubular frame 72. In one example, a center of the third opening 80 may be located equidistance from the first end 72a and the second end 72b of the elongate tubular frame 72. Other suitable locations of the third opening 80 are contemplated. The lumen 84 of the tubular extension 74 may converge with the lumen 82 of the elongate tubular frame 72 at the third opening 80, providing fluid communication therebetween.

The elongate tubular frame 72 may have any suitable configuration. In some examples, the elongate tubular frame 72 may be an elongate tube configured to be implanted or otherwise positioned in a vessel of a patient. Although the elongate tubular frame 72 may be described and depicted as being annular, it is contemplated that the elongate tubular frame 72 may take on any suitable cross-sectional shape. In some examples, the elongate tubular frame 72 may be radially expandable from a first configuration (e.g., a delivery configuration) to a second configuration (e.g., a deployed configuration). Although other configurations are contemplated, the first configuration may be a radially collapsed configuration and the second configuration may be a radially expanded configuration. In some examples, the elongate tubular frame 72 may be deployed to a configuration between a fully radially collapsed configuration and a fully radially expanded configuration.

When the elongate tubular frame 72 is expandable from the first configuration to the second configuration, the elongate tubular frame may be self-expandable. Alternatively or additionally, the expandable elongate tubular frame 72 may be expanded using an expansion mechanism including, but not limited to, a balloon and/or other suitable expanding mechanism.

The elongate tubular frame 72 may take on any suitable form. For example, the elongate tubular frame 72 be a tubular scaffold formed from a tube, one or more filaments, one or more struts, one or more wires, a braided structure, a woven structure, a knitted structure, a cut tube, a stent, and/or the elongate tubular frame 72 may have one or more other suitable forms or structures. In some examples, the elongate tubular frame 72 may have a woven structure fabricated from a number of filaments or struts 36. In some examples, the elongate tubular frame 72 may be knitted or braided with a single filament interwoven with itself and defining open cells. In some examples, the elongate tubular frame 72 may be braided with several filaments interwoven together and may define open cells. A laser cut tubular member forming the elongate tubular frame 72 may have an open and/or closed cell geometry including one or more interconnected filaments or struts defining open cells therebetween.

The elongate tubular frame 72 may be formed from any suitable material. For example, the elongate tubular frame 72 may be formed from a biocompatible material, a biostable material, bioabsorbable material, a biodegradable material, a bioerodible material, a polymer material, a metal material, a radiopaque material, stainless steel, tantalum, tungsten, nickel-titanium alloys such as those possessing shape memory properties commonly referred to as NITINOL, nickel-chromium alloys, nickel-chromium-iron alloys, cobalt-chromium-nickel alloys, and/or other materials. In some examples, the elongate tubular frame 72 may be formed from a superelastic and/or shape memory alloy, such as a self-expanding nickel-titanium alloy. In some examples, the elongate tubular frame 72 may be formed from a radiopaque material. Other suitable materials for the elongate tubular frame 72 are contemplated.

In some examples, one or more radiopaque markers 81 may be applied to (e.g., fixed relative to) the elongate tubular frame 72. In some examples, the one or more radiopaque markers 81 may be at, proximate to, and/or fixed relative to, the third opening 80 to facilitate identifying a location of the third opening 80 and/or the tubular extension 74 through imaging techniques when positioning the vascular access device 70 at the target location or site.

The inner surface 73 and/or the outer surface 75 of the elongate tubular frame 72 may be entirely, substantially, or partially covered with a covering or coating 88. The covering or coating 88 may extend across and/or occlude one or more, or a plurality of the cells defined by the scaffold structure, if any, of the elongate tubular frame 72. Although the coating 88 on the inner surface 73 of the elongate tubular frame 72 is depicted in FIG. 4 as ending prior to the first end 72a of the elongate tubular frame 72, the coating 88 may end at or extend beyond the first end 72a and/or the second end 72b of the elongate tubular frame 72. The covering or coating 88 may seal the elongate tubular frame 72 between the first end 72a and the second end 72b and may facilitate crimping the elongate tubular frame 72 to the first radial configuration and expanding the elongate tubular frame 72 to the second radial configuration. The covering or coating 88 may facilitate mitigation of tissue ingrowth along the elongate tubular frame 72.

The elongate tubular frame 72 may have any suitable length extending from the first end 72a to the second end 72b of the elongate tubular frame 72. In some examples, the length of the elongate tubular frame 72 may be configured extend along the luminal surface of an axillary artery and to anchor within the axillary artery of the patient. Example suitable lengths of the elongate tubular frame 72 may be in a range of 25 millimeters (mm) to 125 mm and/or may be in one or more other suitable ranges. In one example, the length of the elongate tubular frame 72 may be or may be about 100 mm, but other suitable lengths are contemplated.

The first lumen 82 may have any suitable diameter defined by the covering or coating 88 and/or the inner surface 73 of the elongate tubular frame 72 when the elongate tubular frame 72 is in a radially expanded or deployed state. In some examples, a diameter of the first lumen 82 may remain the same along a length of the elongate tubular frame 72 or may differ at one or more longitudinal locations of the elongate tubular frame 72 when compared to a different longitudinal location along the elongate tubular frame 72. In some examples, the first lumen 82 of the elongate tubular frame 72 may be configured to receive and pass therethrough a medical device, such as the blood pump 100. Example diameters of the first lumen 82 may be in a range of 4 mm to 10 mm, a range of 5 mm to 8 mm, and/or one or more other suitable ranges. In one example, the diameter of the first lumen 82 may be or may be about 7 mm, but other suitable diameters are contemplated.

The tubular extension 74 may extend from and/or be coupled with the elongate tubular frame 72 around or proximate to the third opening 80 and/or may extend through the third opening 80. In some examples, the tubular extension 74 collapsible or compressible against the elongate tubular frame 72 and may be configured to extend along the outer surface 75 of the elongate tubular frame 72 when in a delivery configuration and may be configured to extend radially outward from the elongate tubular frame 72 (e.g., radially outward from the outer surface 75 of the elongate tubular frame 72) when in a deployed configuration. In some examples, a second lumen 84 of or defined by the tubular extension 74 may be in fluid communication with the first lumen 82 at or proximate the third opening 80. In some examples, the tubular extension 74 may include a fourth opening 86 that is configured to receive a medical device such that a medical device may be inserted through the second lumen 84, through the third opening 80, and through the first lumen 82 into the vasculature of the patient when the vascular access device 70 is positioned at a target site or location within the vasculature of the patient.

The tubular extension 74 may have any suitable configuration configured to receive a medical device configured to be passed through the vasculature of the patient. In some examples, the tubular extension 74 may be configured to facilitate delivery of the vascular access device 70 to the target location or site in the vasculature of the patient and then once at the target location or site, extend out through the vessel wall of the vessel into extravascular tissue, and through an outer cutaneous layer (e.g., the skin) of the patient for access via a skin incision to insert a medical device therethrough and into the vasculature of the patient. In some examples, the tubular extension 74 may be flexible in a manner that facilitates compressing or collapsing the tubular extension 74 against an outer surface of the elongate tubular frame 72 as the vascular access device 70 is delivered to the target location or site in the vasculature of the patient and adjusting to a deployed configuration or position extending through the outer cutaneous layer of the patient when at the target location or site.

Although not depicted, the tubular extension 74 and/or the third opening 80 may have a seal blocking flow of fluid from the vessel of the patient through the second lumen 84 and out of the fourth opening 86. When included, the seal may be located at any suitable location along the third opening 80 and/or the tubular extension 74 including, but not limited to, at or proximate the fourth opening 86, at or proximate the third opening 80, and/or at one or more other suitable locations between the third opening 80 and the fourth opening 86.

The seal of the tubular extension 74 may be any suitable type of seal. For example, the seal may be a one-way or check seal/valve blocking fluid from exiting the vascular access device 70 via the fourth opening 86, a slit valve or seal, and/or other suitable type of valve or seal.

The tubular extension 74 may have any suitable length configured to extend from the outer surface 75 of the elongate tubular frame 72 positioned within the vascular lumen (e.g., axillary artery) to a location exterior of the outer cutaneous layer of the patient. Example suitable lengths of the tubular extension 74 may be in a range of 25 millimeters (mm) to 125 mm and/or may be in one or more other suitable ranges. In some examples, the length of the tubular extension 74 may be or may be about 80 mm to 120 mm, about 90 mm, or about 100 mm, but other suitable lengths are contemplated.

The second lumen 84 may have any suitable diameter defined by an inner surface of the tubular extension 74 when the tubular extension is in an expanded or deployed state. In some examples, a diameter of the second lumen 84 may remain the same along a length of the tubular extension 74 or may differ at one or more longitudinal locations of the tubular extension 74 when compared to a different longitudinal location along the tubular extension 74. In some examples, the second lumen 84 of the tubular extension 74 may be configured to receive and pass therethrough a medical device, such as the blood pump 100. Example diameters of the second lumen 84 may be in a range of 4 millimeters (mm) to 10 millimeters (mm) a range of 5 mm to 8 mm, and/or one or more other suitable ranges. In one example, the diameter of the second lumen 84 may be or may be about 7 mm, but other suitable diameters are contemplated.

The covering or coating 88, when included, and the tubular extension 74 may be formed from any suitable types of materials. For example, the covering or coating 88 and/or the tubular extension 74 may be a biocompatible material, a flexible material, a polymeric material, a flexible polymer material, a silicone material, a polyurethane material, and/or other suitable type of material. Other suitable polymers include but are not limited to polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In one example, the covering or coating 88 and the tubular extension 74 may be formed from a silicone material.

The covering or coating 88 and the tubular extension 74 may be configured in any suitable manner with respect to one another. The covering or coating 88 and the tubular extension 74 may be formed from the same material or different materials. In some examples, the covering or coating 88 and the tubular extension 74 may be monolithically formed from a single material. In some examples, the covering or coating 88 and the tubular extension 74 may be continuously formed from a single material. In some examples, the covering or coating 88 and the tubular extension 74 may be formed from the same or different materials and coupled together. In some examples, the covering or coating 88 and the tubular extension 74 may be formed from the same or different materials and may be separate components not coupled to one another. Other suitable configurations of the covering or coating 88 and the tubular extension 74 are contemplated.

In some instances, the tubular extension 74 may be devoid of any tubular scaffold, framework or reinforcement structure, such that the tubular extension 74 is formed entirely of a flexible polymer material (e.g., silicone). Thus, in some instances the tubular extension 74 may consist of a flexible polymer material without any tubular scaffold extending therealong and/or embedded therein. The flexible polymer material may permit the tubular extension 74 to be readily deformed, flexed and/or deflected to manipulate the tubular extension 74 during placement of the vascular access device 70.

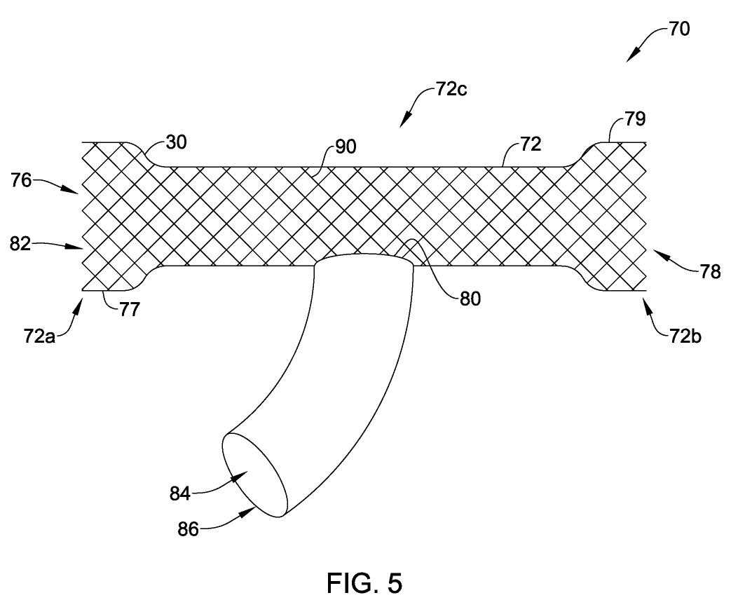

FIG. 5 depicts a schematic side view of an illustrative stent assembly configuration of the vascular access device 70. The configuration of the vascular access device 70 depicted in FIG. 5 may include the elongate tubular frame 72 configured as a stent. In some examples, the elongate tubular frame 72 configured as a stent may be configured as a self-expanding stent, but other suitable stents are contemplated. The tubular extension 74 may extend from the third opening 80 of the elongate tubular frame 72 and may be formed from a flexible polymer, which may permit adjusting the angle and/or curvature of the tubular extension 74 extending from the elongate tubular frame 72 in order to facilitate directing the tubular extension 74 through extravascular tissue to a skin incision. Although not depicted in FIG. 5, the vascular access device 70 may include the coating 88, but configurations are contemplated in which the coating 88 is omitted.

The elongate tubular frame 72 having a tubular scaffold configured as a stent may include, among other features discussed herein, the first end 72a, the second end 72b, the intermediate region 72c, the first opening 76, the second opening 78, and the third opening 80. The first lumen 82 may extend from the first opening 76 through to the second opening 78 and may be in fluid communication with the third opening 80.

The elongate tubular frame 72 configured as a stent or stent-like structure may have any suitable stent structure. Example suitable stent structures include, but are not limited to, a woven structure fabricated from a number of filaments or struts 90, a braided structure from one filament or struts 90, a braided structure from several filaments, a knitted structure from one or more filaments or struts 90, a knotted structure from one or more filaments or struts 90, a laser cut tubular structure defining one or more interconnected filaments or struts 90, and/or other suitable stent structures discussed herein or otherwise.

As depicted in FIG. 5, in the expanded configuration, the elongate tubular frame 72 configured as a stent may include retention features or anti-migration flared regions 77, 79 positioned adjacent to the first end 72a and the second end 72b of the elongate tubular frame 72, respectively. The anti-migration flared regions 77, 79 may be configured to engage a luminal surface of the wall of the vessel at the target location or site in the vasculature. In some examples, the retention features or flared regions 77, 79 may have a larger diameter than an intermediate region 72c of the elongate tubular frame 72 to prevent the elongate tubular frame from migrating once placed in the vessel. It is contemplated that the transition from the cross-sectional area of the intermediate region 72c to the retention features or flared regions 77, 79 may be gradual, sloped, or occur in an abrupt step-wise manner, as desired. Although the retention features or flared regions 77, 79 are described with respect to the elongate tubular frame 72 configured as a stent, other non-stent configurations of the elongate tubular frame 72 may incorporate the retention features or flared regions 77, 79 or at least one or more the functions thereof.

The first anti-migration flared region 77 may have a first outer diameter and the second anti-migration flared region 79 may have a second outer diameter. In some instances, the first and second outer diameters may be approximately the same, while in other instances, the first and second outer diameters may be different. In some examples, the elongate tubular frame 72 may include only one or none of the anti-migration flared regions 77, 79. For example, the first end 72a may include the anti-migration flared region 77 while the second end 72b may have an outer diameter similar to the intermediate region 72c. It is further contemplated that the second end 72b may include the anti-migration flared region 79 while the first end 72a may have an outer diameter similar to an outer diameter of the intermediate region 72c. In some examples, the elongate tubular frame 72 may have a uniform outer diameter from the first end 72a to the second end 72b. In some examples, the outer diameter of the intermediate region 72c may be in the range of 5 mm to 10 mm. The outer diameter of the anti-migration flared regions 77, 79 may be in the range of 7 mm to 12 mm. It is contemplated that the outer diameter of the elongate tubular frame 72 may be varied to suit the desired application.

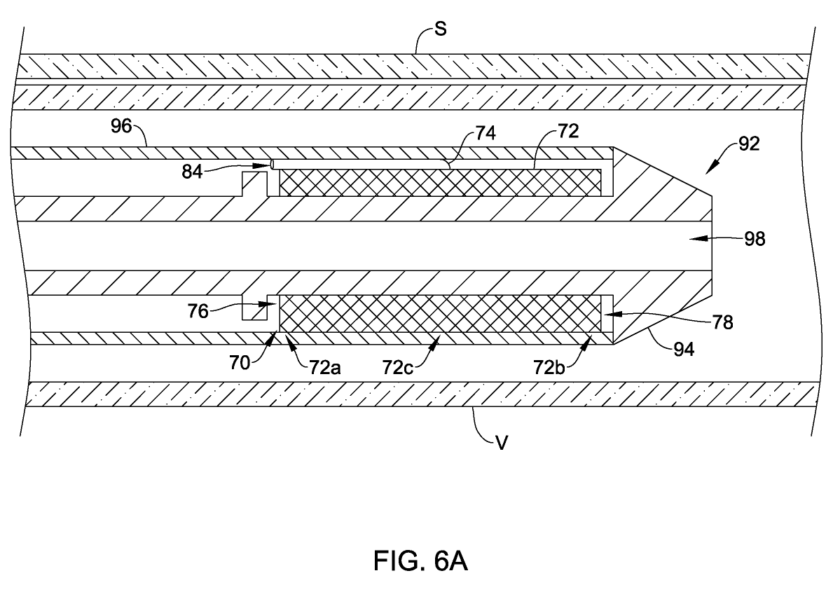

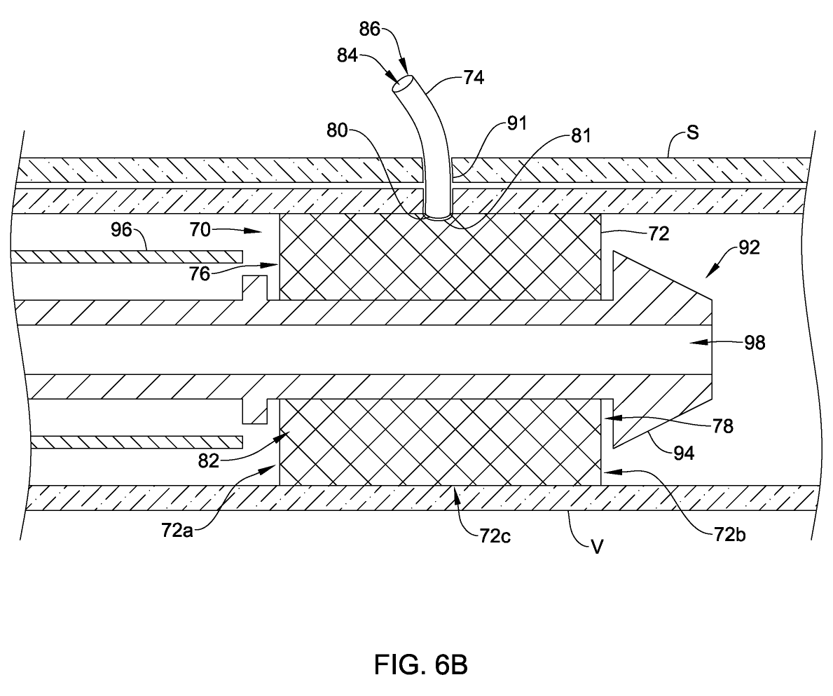

FIGS. 6A and the 6B depict schematic cross-section views of an illustrative configuration of the vascular access device 70 along an illustrative configuration of a delivery assembly 92 having an inner shaft 94 and an outer sheath 96. In some examples, the inner shaft 94 may include or define a guidewire lumen 98 to facilitate tracking a guidewire to the target location. The delivery assembly 92 may include additional and/or alternative features. The delivery assembly 92 is depicted with the vascular access device 70 in a vessel V proximate the skin S (e.g., the outer cutaneous layer). However, in some examples, the vessel V may be closer to or farther away from the skin S.

FIG. 6A schematically depicts the vascular access device 70 positioned on the inner shaft 94 in a first, radially constrained configuration (e.g., a delivery configuration). When on the inner shaft 94 and in the first configuration, the outer sheath 96 may compress the elongate tubular frame 72 and/or the tubular extension 74 on the inner shaft 94 or otherwise facilitate maintaining the vascular access device 70 in the first configuration.

When the vascular access device 70 is in the first configuration, the tubular extension 74 may be at any suitable location. In some examples, when the vascular access device 70 is in the first configuration, the tubular extension 74 may be compressed or otherwise positioned along the outer surface 75 (not labeled in FIG. 6A) of the elongate tubular frame 72, as depicted in FIG. 6A. In some examples, the tubular extension 74 may be located within the first lumen 82 of the elongate tubular frame 72. Other suitable positioning of the tubular extension 74 is contemplated when the vascular access device 70 is in the first configuration.

FIG. 6B schematically depicts the vascular access device 70 positioned in a second configuration (e.g., a deployed configuration) at a target location or site in the vessel V. As depicted in FIG. 6B, the outer sheath 96 has been advanced in a proximal direction (e.g., withdrawn). When the elongate tubular frame 72 is a self-expanding component (e.g., a self-expanding stent, etc.), the elongate tubular frame 72 may automatically adjust (e.g., automatically radially expand) from the first configuration to the second configuration in response to moving the outer sheath 96 in the proximal direction relative to the vascular access device 70 and/or the inner shaft 94 to uncover and unconstrain the elongate tubular frame 72.

Before, during, or after advancing the outer sheath 96 in the proximal direction after the vascular access device 70 in the delivery assembly 92 has reached the target location or site, the inner shaft 94 and/or the delivery assembly 92 may be rotated to position the tubular extension 74 proximate the skin S. In some examples, the vascular access device 70 may be imaged and a practitioner may observe the radiopaque marker 81 proximate the third opening 80 and position the radiopaque marker 81 proximate or facing the skin S where access to the vascular access device 70 is desired. Once the vascular access device 70 is positioned, the outer sheath 96 may be withdrawn such that the elongate tubular frame 72 may adjust (e.g., radially expand) from the first configuration to the second configuration.

When the elongate tubular frame 72 has adjusted from the first configuration to the second configuration with the third opening 80 facing or proximate the skin S of the patient, an incision 91 may be formed through the skin S (such as a subscapular location) and the vascular wall of the vessel V at or proximate and radially outward from the third opening 80 of the elongate tubular frame 72. Once the incision 91 is made, the tubular extension 74 may be located and pulled or otherwise passed out through the vascular wall of the vessel V, through extravascular tissue, and out through the incision 91. In some examples, a tweezers or forceps may be utilized to grasp the tubular extension 74 within the vessel V and pull the tubular extension through the vascular wall of the vessel V, through the extravascular tissue, and to the skin S via the incision 91.

Once the tubular extension 74 is extending through the incision 91 and accessible to the exterior of the patient, the medical device may be inserted into the vessel V through the second lumen 84 along the tubular extension 74 and the first lumen 82 along the elongate tubular frame 72 and delivered to a target site or location (e.g., the heart of the patient). Although the patient may naturally clot around the tubular extension 74, the skin S of the patient may be sutured around the tubular extension extending through the incision 91.

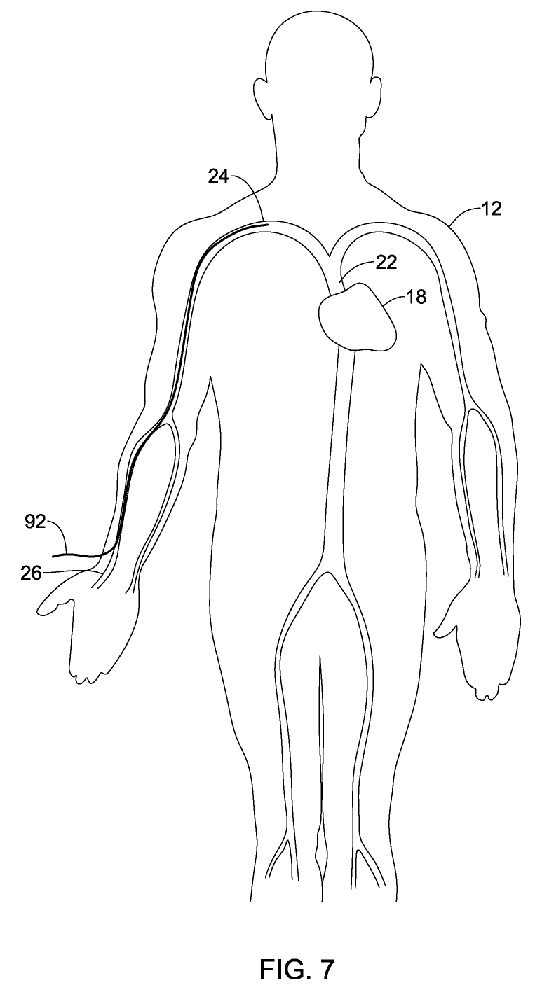

FIG. 7 schematically depicts a patient 12, with vasculature and the heart 18 of the patient 12 shown. The depicted vasculature of the patient includes, the aorta 22 extending from the heart 18, the axillary artery 24, and the radial artery 26. In some examples, the delivery assembly 92 may access the vasculature of the patient 12 at the radial artery 26 near a hand 89 of the patient 12 and once in the vasculature of the patient 12, the delivery assembly 92 may traverse the vasculature to a target location or site at or along the axillary artery 24 (e.g., a right axillary artery), as depicted for example in FIG. 7. In some examples, the target location or site may be located at or proximate a subscapular artery extending from the axillary artery 24, near a junction between the second and third part of the axillary artery 24. Although the target location or site for implanting the vascular access device 70 is discussed as being in the axillary artery 24, other suitable upper extremity access points may be utilized that facilitate coupling the elongate shaft 50 extending from the blood pump 100 with a power or control source and/or that facilitates the patient 12 being ambulatory with the blood pump 100 operational within the heart 18.

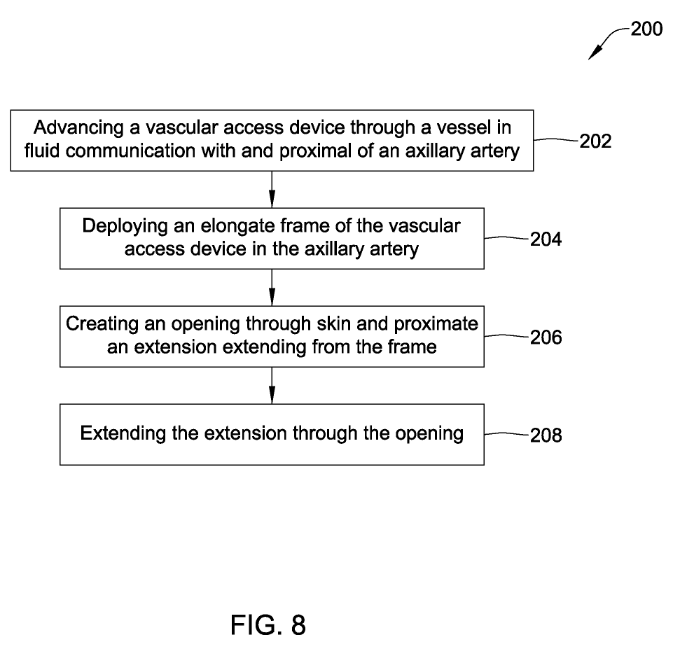

FIG. 8 depicts a schematic box diagram of an illustrative technique or method 200 of using a vascular access device. The method 200 of using the vascular access device may utilize the vascular access device configurations discussed herein and/or other suitable vascular access device configurations.

The method 200 may include advancing 202 a vascular access device through a percutaneous access site at a vessel in fluid communication with and proximal of an axillary artery. Although other suitable vessels may be utilized for percutaneous access to the axillary artery, an example of a vessel in fluid communication with and proximal of the axillary artery at which a percutaneous access site may be located is the radial artery near a hand of the patient. In some examples, the vascular access device may be in a first configuration (e.g., a delivery configuration) when in a delivery assembly and passed through the radial artery into the axillary artery. In some examples, a guidewire may be extended through the access site and to the target location or site, such that the delivery assembly may be passed over the guidewire. The vascular access device advanced through the vessel may have any suitable configuration including, but not limited to, an elongate tubular frame and a tubular extension extending radially outward from an outer surface of the elongate tubular frame, as discussed herein or otherwise.

The method 200 may include deploying 204 the elongate tubular frame of the vascular access device in the lumen of the axillary artery of the patient. For example, once the vascular access device has been positioned at the target location or site within the axillary artery or other suitable vessel, an outer sheath of the delivery assembly made be withdrawn in a proximal direction so as to uncover all of or at least part of the vascular access device. The elongate tubular frame of the vascular access device may then expand radially outward to a second configuration (e.g., a deployed configuration) and exert a radially outward force against the luminal surface of the vascular wall, either through self-expansion or through a user actuating the elongate tubular frame to expand and anchor in the vessel.

In some cases, prior to deploying 204 the elongate tubular frame in the vessel, the delivery assembly and/or the vascular access device may be oriented at the target site or location (e.g., a desired location) relative to an intended location of an opening through or incision in the skin and/or axillary artery. In some examples, the vascular access device and/or the delivery assembly may be rotated to ensure a tubular extension of the vascular access device is facing a side of the vessel that is closest to the skin of the patient where a skin incision is desired to be formed for accessing the vascular access device. The positioning of the tubular extension may be confirmed through identifying features of the vascular access device including, but not limited to, radiopaque markers observed via visualization techniques.

The method 200 may include creating 206 an opening or incision through skin of the patient at a location proximate the tubular extension extending from the elongate tubular frame of the vascular access device. The opening through the skin of the patient may be made using a surgical knife (e.g., scalpel) and/or other suitable tool and may extend through extravascular tissue, and through the vascular wall of the axillary artery or other vessel of the patient.

The method 200 may include extending 208 the tubular extension of the vascular access device out of the incision in the vascular wall of the artery, through the extravascular tissue, and out through the opening in the skin (e.g., skin incision) of the patient. The tubular extension may be grasped using a tweezers, forceps, and/or other suitable grasper and pulled out through the wall of the vessel and/or the skin of the patient. In some examples, the opening may be stitched around or to the tubular extension to mitigate bleeding and/or a likelihood of an infection.

Once the tubular extension is extending through the skin of the patient, a medical device (e.g., a blood pump, etc.) may be inserted into the tubular extension, through the elongate tubular frame and into vasculature of the patient. Once in the vasculature, the medical device may be delivered to a target location or site (e.g., the heart and/or the aorta of the patient, etc.)

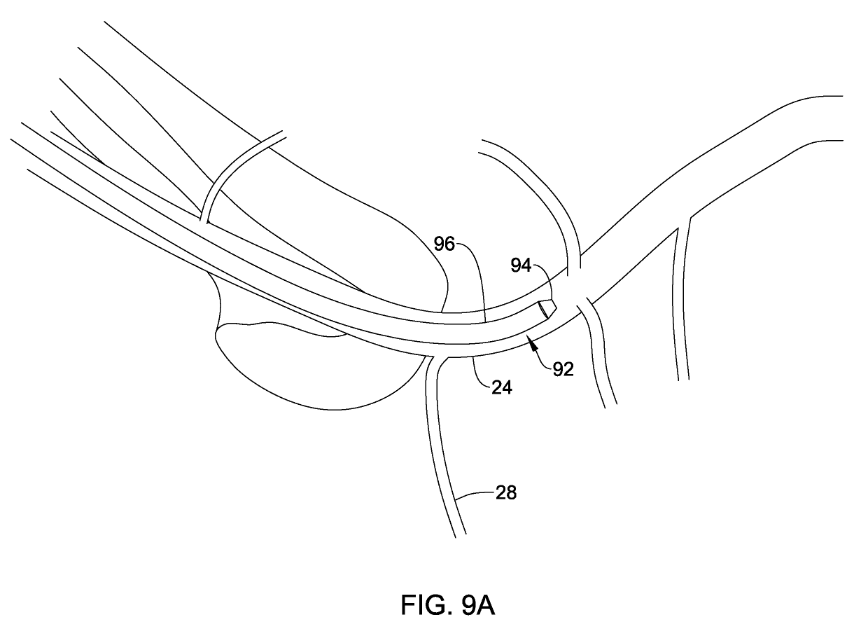

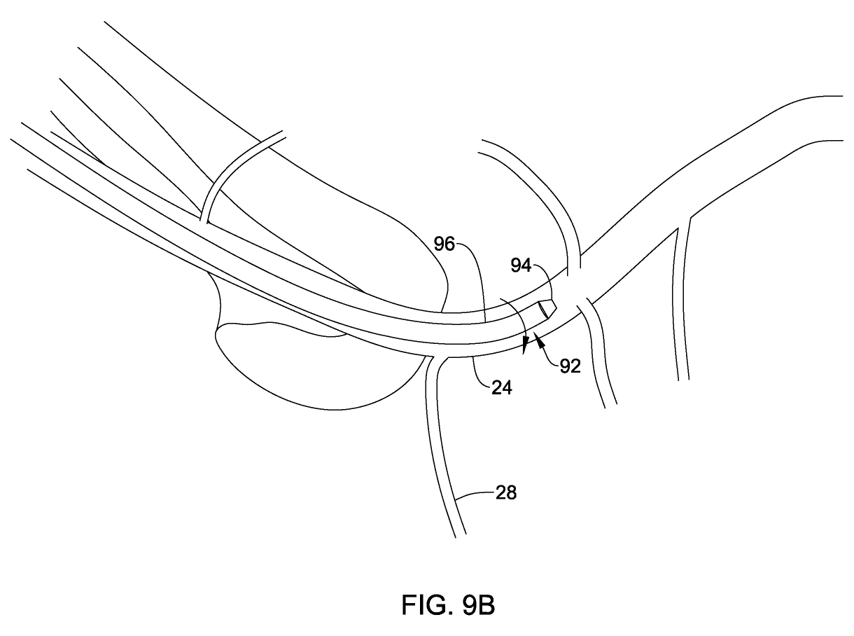

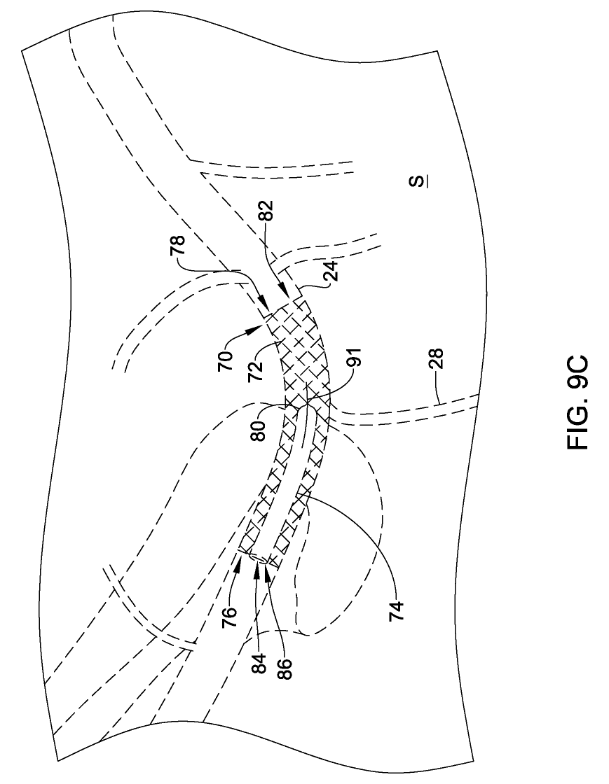

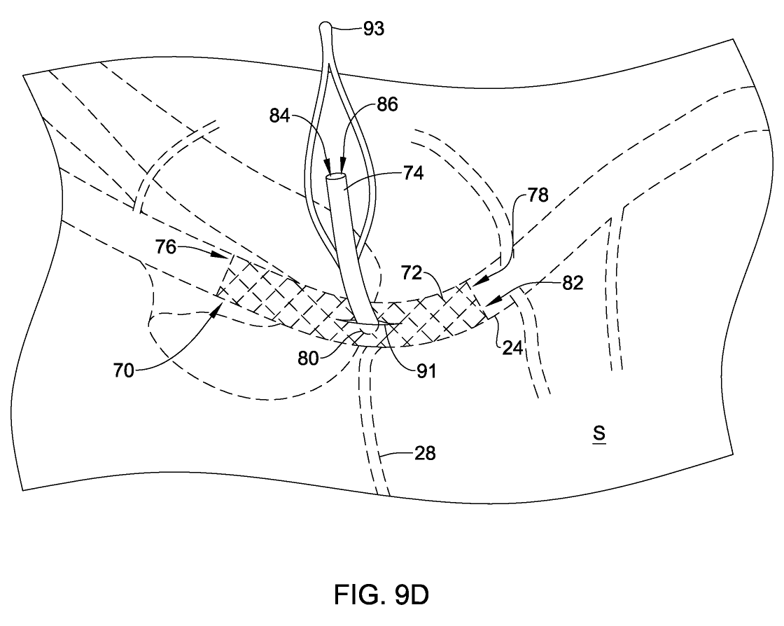

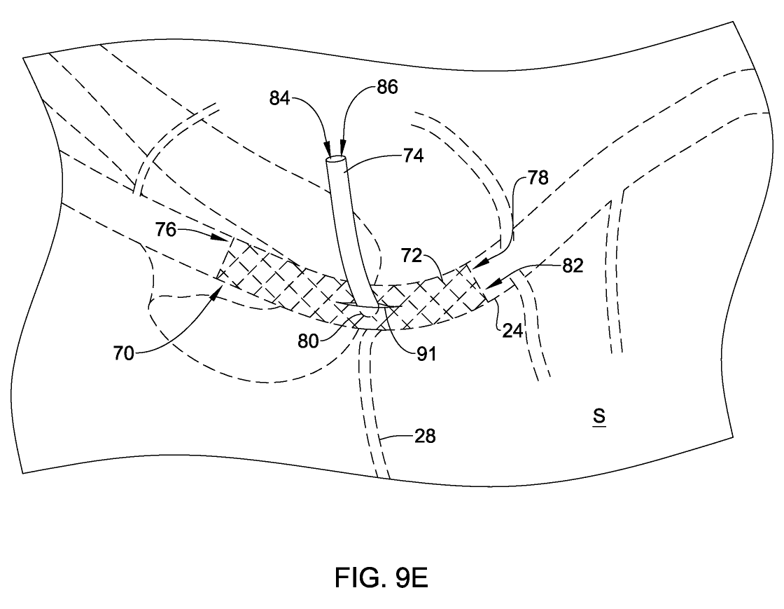

FIGS. 9A-9E schematically depict illustrative steps in a technique for positioning the vascular access device 70 at a target location or site, such as the axillary artery 24. FIGS. 9A and 9B depict vasculature of the patient, including the axillary artery 24 and the subscapular artery 28. FIGS. 9C-9E depict the vasculature of the patient show in FIGS. 9A and 9B in broken lines behind a layer of skin S.

FIG. 9A schematically depicts the delivery assembly 92 having the inner shaft 94 and the outer sheath 96 delivered to the axillary artery 24 proximate the subscapular artery 28. As discussed herein, the axillary artery 24 and/or other upper extremity access locations through which a medical device, such as the blood pump 100 or other suitable medical device, may be delivered to the vasculature of the patient, may be accessed via a percutaneous access point. In some examples, the percutaneous access point may access the radial artery of the patient at or proximate the hand or forearm of the patient.

FIG. 9B schematically depicts orienting the delivery assembly 92 (and the vascular access device 70 therein and not depicted in FIG. 9B). To orient the delivery assembly 92, a practitioner may visualize the delivery assembly 92 and vascular access device 70 therein on a screen or one or more images and rotate and/or axially adjust the delivery assembly 92 until the third opening 80 and/or the tubular extension 74 is at a desired location relative to the skin of the patient. For example, the delivery assembly 92 may be rotated until the third opening 80 and/or the tubular extension 74 are facing a wall of the axillary artery 24 nearest the skin of the patient where a skin incision is desired for accessing the vascular access device 70.

FIG. 9C schematically depicts the elongate tubular frame 72 of the vascular access device 70 deployed in the axillary artery 24 across a junction with the subscapular artery 28, but other suitable locations for elongate tubular frame 72 are contemplated. The delivery assembly 92 has been withdrawn from the vasculature and is not depicted in FIG. 9C, but it is contemplated the delivery assembly 92 may remain proximate the vascular access device 70 in the event the vascular access device 70 may need to be recaptured and/or reoriented. As depicted in FIG. 9C, the vasculature of the subject and the vascular access device 70 are shown in broken lines to represent such features as being behind a layer of skin.

The third opening 80 and the tubular extension 74 have been oriented toward the skin S in FIG. 9C. The incision 91 through the skin S (e.g., an incision in a scapula/clavicle/shoulder region of the patient) and through the vascular wall of the vessel may be formed proximate the third opening 80 to facilitate extending the tubular extension out through the vascular wall of the vessel, through extravascular tissue, and out through the incision 91.

FIG. 9D schematically depicts grasping the tubular extension 74 with a grasper 93 (e.g., a tweezers, a forceps, fingers, and/or other suitable grasper) with the tubular extension 74 extending outward from the axillary artery 24 through extravascular tissue and through the incision 91. In operation, after the incision 91 is formed through the skin S and the wall of the vessel (e.g., the axillary artery 24), a practitioner may insert the grasper 93 through the incision 91 and into the lumen of the vessel to grasp the tubular extension with the grasper 93. The practitioner may then pull the tubular extension 74 out through the wall of the vessel, through the extravascular tissue, and out through the incision 91. Other suitable techniques for extending the tubular extension 74 through the incision such that the tubular extension 74 extends outward from the vascular wall of the vessel through extravascular tissue to the incision are contemplated. In some examples, the incision 91 may be closed around the tubular extension 74 using sutures, glue, and/or other closure techniques.

FIG. 9E schematically depicts the vascular access device 70 at the target location or site and the tubular extension 74 fully extending from the vessel through extravascular tissue to the incision 91. With the tubular extension 74 extending through the incision 91 and the lumen 84 of the tubular extension 74 accessible from exterior of the patient, the medical device (e.g., blood pump 100 of the percutaneous circulatory support device 10) may be inserted through the fourth opening 86, into the second lumen 84 of the tubular extension 74, into the first lumen 82, and into the vasculature of the patient. Once in the vasculature of the patient, the medical device (e.g., the blood pump 100) may be delivered to a target location (e.g., at the aorta and/or heart of the patient). Further, an elongate shaft or power cord (e.g., the elongate shaft 50 of the percutaneous circulatory support device 10) extending from the medical device (e.g., the blood pump 100) may extend out of the tubular extension 74 such that the medical device may be coupled to a portable power source or control source, while the patient is ambulatory.

Additional and/or alternative steps to those described with respect to FIGS. 8-9E may be utilized to deliver the vascular access device 70 to a target site. Similarly, additional and/or alternative steps to those described may be utilized to deliver the blood pump 100 and/or other suitable medical devices to the target location within anatomy of the patient via the vascular access device 70.

The materials that can be used for the various components of the devices, assembly, systems, and the various elements thereof disclosed herein may include those commonly associated with medical devices. In some instances, the devices described herein, and/or components thereof, may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, ElastEon® from Aortech Biomaterials or ChronoSil® from AdvanSource Biomaterials), biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some instances, portions or all of the devices, assemblies, and/or systems described herein, and/or components thereof, may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the apparatus in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the apparatus to achieve the same result.

In some instances, a degree of Magnetic Resonance Imaging (MRI) compatibility may be imparted into the devices and/or other elements disclosed herein. For example, the devices described herein, and/or components or portions thereof, may be made of a material that does not substantially distort the image and create substantial artifacts (e.g., gaps in the image). Certain ferromagnetic materials, for example, may not be suitable because they may create artifacts in an MRI image. The devices, assembly, and/or systems described herein, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example configuration being used in other configurations. The scope of the disclosure is, of course, defined in the language in which the appended claims are expressed.

What is claimed is:

1. A vascular access device comprising:
an elongate tubular frame defining a first opening at a first end of the elongate tubular frame, a second opening at a second end of the elongate tubular frame, and a third opening at a location between the first end and the second end, wherein a first lumen extending through the elongate tubular frame is in communication with the first opening, the second opening, and the third opening; and
a tubular extension formed from a flexible polymer and defining a second lumen in fluid communication with the first lumen at the third opening, the tubular extension extends radially outward from an outer surface of the elongate tubular frame.

2. The vascular access device of claim 1, wherein the elongate tubular frame is a stent.

3. The vascular access device of claim 1, wherein the elongate tubular frame is a self-expanding elongate tubular frame.

4. The vascular access device of claim 1, further comprising:
a coating extending along an inner surface of the elongate tubular frame and defining the first lumen extending through the elongate tubular frame.

5. The vascular access device of claim 4, wherein the coating and the tubular extension are monolithically formed from a single material.

6. The vascular access device of claim 4, wherein the coating and the tubular extension are formed from silicone.

7. The vascular access device of claim 1, further comprising:
a radiopaque marker fixed relative to the third opening.

8. The vascular access device of claim 1, wherein the elongate tubular frame is formed from a radiopaque material.

9. The vascular access device of claim 1, wherein the tubular extension has a length in a range of 25.0 millimeters (mm) to 125.0 mm.

10. The vascular access device of claim 1, wherein one or both of the first lumen and the second lumen have a maximum diameter in a range 5.0 mm to 8 mm.

11. The vascular access device of claim 1, wherein a center of the third opening is equidistance from the first end of the elongate tubular frame and the second end of the elongate tubular frame.

12. A method of using a vascular access device, the method comprising:
advancing the vascular access device through a vessel in fluid communication with and proximal of an axillary artery, the vascular access device having an elongate tubular frame and a tubular extension extending radially outward from an outer surface of the elongate tubular frame;
deploying the elongate tubular frame within the axillary artery with the tubular extension extending along the elongate tubular frame;
creating an opening through skin and at a location proximate the tubular extension; and
extending the tubular extension through the opening.

13. The method of claim 12, further comprising:
orienting the tubular extension at a desired location relative to an intended location of the opening through the skin and the axillary artery.

14. The method of claim 12, further comprising:
delivering a blood pump to a heart through the tubular extension and the elongate tubular frame.

15. The method of claim 12, further comprising:
percutaneously accessing the vessel in fluid communication with and proximal of the axillary artery.

16. The method of claim 12, wherein the vessel is a radial artery.

17. The method of claim 12, wherein the tubular extension is formed from a flexible polymer material.

18. A stent assembly comprising:
an elongate tubular frame having an inner surface and an outer surface, the elongate tubular frame defining a first opening at a first end of the elongate tubular frame, a second opening at a second end of the elongate tubular frame, and a third opening extending through the elongate tubular frame at a location between the first end and the second end;
a coating extending along the inner surface of the elongate tubular frame and defining a first lumen extending through the elongate tubular frame; and
a tubular extension defining a second lumen in fluid communication with the first lumen at the third opening.

19. The stent assembly of claim 18, wherein the coating extending along the inner surface of the elongate tubular frame and the tubular extension are continuously formed from a flexible polymer material.

20. The stent assembly of claim 18, wherein the elongate tubular frame has a minimum outer diameter that is less than 5.0 mm and a maximum outer diameter that is less than 8.0 mm.
